(19) United States
(12) Patent Application Publication
(10) Pub. No.: US 2023/0329602 A1
(43) Pub. Date: Oct. 19, 2023

(54) METHOD FOR PREPARING A COUNTER/REFERENCE ELECTRODE

(71) Applicant: Roche Diabetes Care, Inc., Indianapolis, IN (US)

(72) Inventors: Kirill Sliozberg, Mannheim (DE); Alexander Steck, Hirschberg (DE)

(21) Appl. No.: 18/322,335

(22) Filed: May 23, 2023

Related U.S. Application Data
(63) Continuation of application No. PCT/EP2021/082392, filed on Nov. 22, 2021.

(30) Foreign Application Priority Data
Nov. 24, 2020 (EP) 20 209 558.4

(51) Int. Cl. A61B 5/1486

(52) U.S. Cl. CPC A61B 5/14865

(57) ABSTRACT

This disclosure generally relates to a method for the preparation of an electrode and to an analyte sensor having the electrode as well as to the use of the analyte sensor for detecting an analyte in a sample. In particular, this disclosure relates to a method for the preparation of an electrode in which Ag+ cations present in the electrode material are at least partially reduced.

### RELATED APPLICATIONS

This application is a continuation of International Patent Application No. PCT/EP2021/082392, filed Nov. 22, 2021, which claims priority to EP 20 209 558.4, filed Nov. 24, 2020, both of which are hereby incorporated herein by reference.

### BACKGROUND

This disclosure teaches a method for the preparation of an electrode and to an analyte sensor comprising the electrode as well as to the use of the analyte sensor for detecting at least one analyte in a sample. In particular, this disclosure teaches a method for the preparation of an electrode, the method comprising a partial reduction of Ag⁺ cations present in the electrode material.

Monitoring certain body functions, more particularly monitoring one or more concentrations of certain analytes, plays an important role in the prevention and treatment of various diseases.

Along with so-called point measurements in which a sample of a body fluid is specifically taken from a user and investigated for the analyte concentration, continuous measurements are increasingly becoming available. Hence, there is an increasing demand for accurate analyte sensors that enable reliable and cost-efficient analyte detection from a body fluid or other samples. An analyte sensor for determining the concentration of an analyte under in vivo conditions is known from WO 2010/028708 A1. Another example of such sensor is disclosed in WO 2012/130841 A1. Moreover, WO 2007/147475 A1 discloses an amperometric sensor configured for implantation into a living body to measure the concentration of an analyte in a body fluid. An alternative sensor element is disclosed in WO 2014/001382 A1.

AgCl is frequently used as a constituent part of a reference or counter/reference electrode in a subcutaneous electrochemical sensor. The AgCl-containing electrode is typically made by coating of a substrate with a paste or ink consisting of AgCl, a binder and optionally further components, in particular elemental silver.

There are, however, disadvantages associated with the use of an AgCl-containing electrode. After implantation of a sensor into a user's body, the AgCl on the outer surface of the electrode comes into contact with physiological components of the interstitial fluid (ISF), whereby the substantially insoluble AgCl may be converted into soluble Ag-containing compounds which diffuse towards the working electrode of the sensor. A high local concentration of soluble Ag-containing compounds in the vicinity of an enzyme-based working electrode such as glucose dehydrogenase (GOD) may lead to a reversible or even irreversible deactivation of the enzyme. Another major problem of the release of soluble Ag-containing compounds from the electrode surface is a loss of biocompatibility since these compounds are known to be highly cytotoxic.

A further possible disadvantage is a chemical reaction of AgCl with compounds found in the ISF, possibly including glucose, which results in local glucose consumption and, thus, an alteration of the local glucose concentration, which may lead to inaccurate glucose detection.

A still further disadvantage is an effect possibly caused by an immune response, which results in an alteration of the local ISF composition and, again, to an inaccurate analyte detection, which may influence the measurement.

Thus, there is a need to reduce the release and/or accessibility of AgCl from an electrode of an in vivo analyte sensor. A possible approach would be a general reduction of the AgCl content in the electrode material. This solution, however, is incompatible with the need of a sufficient AgCl content for a proper sensor function.

A further approach to avoid a possible poisoning of an enzyme-based working electrode is increasing the distance between the working electrode and the reference or counter/reference electrode. Nevertheless, this approach is not applicable for sensors with limited available space and further does not solve the biocompatibility issue and possible side-reactions with components of ISF.

EP 3 308 152 B1 discloses a method for generating a layer of AgCl at a surface of an electrode of a sensor wherein a sensor material consisting of elemental Ag is provided and AgCl is formed by oxidation of silver metal.

U.S. Pat. No. 8,620,398 B2 discloses a method for regenerating a reference electrode of a sensor by inverting the applied potential during use.

X. Jin et al., Journal of electroanalytical chemistry 542 (2003), 85-96 discloses the manufacturing of an AgCl electrode. In this method AgCl is reduced to Ag.

U.S. Pat. No. 5,565,143 relates to silver/silver chloride polymer compositions for use in making electrodes.

### SUMMARY

This disclosure teaches methods for preparing an AgCl-containing electrode and an analyte sensor, which address the above-mentioned technical challenges. An AgCl-containing electrode and an analyte sensor, which provide a decreased release and/or accessibility of Ag-containing compounds while retaining a proper sensor function, are disclosed.

The method according to this disclosure is advantageous as it allows the preparation of an AgCl-containing electrode, which may be comprised in an analyte sensor with a reduced leakage and/or accessibility of AgCl, thereby allowing a stable sensor function without poisoning of the enzyme-containing working electrode and without cytotoxicity problems for the user.

According to this disclosure, a method for the preparation of an AgCl-containing electrode on a substrate is disclosed. The AgCl-containing electrode may be part of an analyte sensor.

The method comprises the following steps, which may be performed in the given order. Further, if not indicated otherwise, two or more process steps may be performed simultaneously or partially simultaneously. Further, one or more than one or even all of the method steps may be performed once or more than once or even repeatedly or continuously. The method may further comprise additional method steps, which are not listed specifically.

A first aspect of this disclosure relates to a method for manufacturing an electrode of an analyte sensor, the method comprising the steps:
a) providing a substrate comprising
   a first side and a second side, and
   at least one conductive material positioned on the first side of the substrate,
b) applying a layer of an AgCl-containing composition onto the conductive material, wherein the layer of the AgCl-containing composition comprises an outer surface and an inner surface, wherein the outer surface faces away from the conductive material and wherein the inner surface is in contact with the conductive material, and
c) at least partially reducing AgCl on the outer surface of the layer of the AgCl-containing composition, thereby forming elemental Ag on the outer surface, and
d) obtaining the electrode of the analyte sensor on the first side of the substrate.

In particular embodiments, the electrode is a counter electrode and/or a reference electrode and/or a combined counter/reference electrode of an analyte sensor.

A further aspect of this disclosure relates to a method for manufacturing an analyte sensor comprising manufacturing the electrode as described above and providing at least one working electrode.

A further aspect of this disclosure relates to an electrode of an analyte sensor obtainable by the method as described above.

A further aspect of this disclosure relates to an analyte sensor obtainable by the method as described above.

A further aspect of this disclosure relates to an analyte sensor comprising:
(i) a substrate comprising
   a first side and a second side, and
   at least one conductive material positioned on the first side of the substrate,
(ii) an electrode positioned on the at least one conductive material, wherein the electrode comprises a layer of an AgCl-containing composition comprising an outer surface and an inner surface, wherein the outer surface faces away from the conductive material and wherein the inner surface is in contact with the conductive material, and wherein the AgCl on the outer surface of the layer of the AgCl-containing composition is at least partially reduced and elemental Ag is present on the outer surface of the AgCl-containing composition, and
(iii) at least one working electrode.

Still a further aspect of this disclosure relates to an analyte sensor comprising an electrode as described above and at least one working electrode.

As used in the following, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation, in which one or more further features are present. As an example, the expressions “A has B,” “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

Further, it shall be noted that the terms “at least one,” “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once. It shall also be understood for purposes of this disclosure and appended claims that, regardless of whether the phrases “one or more” or “at least one” precede an element or feature appearing in this disclosure or claims, such element or feature shall not receive a singular interpretation unless it is made explicit herein. By way of non-limiting example, the terms “electrode,” “conductive material,” “binder” and “enzyme,” to name just a few, should be interpreted wherever they appear in this disclosure and claims to mean “at least one” or “one or more” regardless of whether they are introduced with the expressions “at least one” or “one or more.” All other terms used herein should be similarly interpreted unless it is made explicit that a singular interpretation is intended.

Further, as used in the following, the terms “preferably,” “more preferably,” “particularly,” “more particularly,” “specifically,” “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

This disclosure relates to a method for manufacturing of an electrode of an analyte sensor as described above and to an electrode as described above. The electrode is manufactured outside a user's body, i.e., before the electrode, in particular, the analyte sensor, is implanted into a user's body.

The electrode of this disclosure is an electrode comprising an AgCl-containing composition comprised in an analyte sensor. Typically, the electrode is a counter electrode, and/or a reference electrode and/or a combined counter/reference electrode.

In addition, this disclosure discloses a method for manufacturing an analyte sensor. The method for manufacturing of an analyte sensor comprises the method for manufacturing an electrode on a substrate as disclosed herein and a step of providing at least one working electrode. The analyte sensor is manufactured outside a user's body, i.e., before the analyte sensor is implanted into a user's body.

The analyte sensor may be configured for at least partial implantation, specifically transcutaneous insertion, into a body tissue of a user; more specifically the analyte sensor may be configured for continuous monitoring of the analyte, even more specifically the analyte sensor may be configured for continuous glucose monitoring. In certain embodiments, the analyte sensor is sterilized and/or packaged after its manufacturing.

The terms “user” and “subject” are used interchangeably herein. The terms may in particular relate to a human being.

The term “analyte sensor” as used herein is a broad term and is to be given its ordinary and customary meaning to a person of ordinary skill in the art and is not to be limited to a special or customized meaning. The term specifically may refer, without limitation, to an arbitrary element or device configured for detecting or for measuring the concentration of the at least one analyte. The analyte sensor specifically may be an analyte sensor suitable for at least partial implantation into a body tissue of a user, more specifically an analyte sensor for continuous monitoring of the analyte.

In particular embodiments, the analyte sensor of this disclosure is an electrochemical sensor comprising at least one working electrode, at least one further electrode and respective circuitry. More particularly, the sensor is an amperometric electrochemical sensor comprising at least one working electrode and the at least one AgCl-containing electrode of this disclosure, which may be a counter electrode and/or a reference electrode or a combined counter/reference electrode.

Step (a) of the method of this disclosure comprises the steps of providing a substrate comprising a first side and a second side, and at least one conductive material positioned on the first side of the substrate.

The term “substrate” as used herein, is a broad term and is to be given its ordinary and customary meaning to a person of ordinary skill in the art and is not to be limited to a special or customized meaning. The term “substrate” specifically may refer, without limitation, to any kind of material or combination of materials, which is suitable to form a carrier layer to support the conductive material, the layer of an AgCl-containing composition and/or the layer of sensing material as described herein. In particular, a “substrate” as understood herein may comprise electrically insulating material. In certain embodiments, the substrate may be a sheet, a roll or a plate.

The term “electrically insulating material,” as used herein, is a broad term and is to be given its ordinary and customary meaning to a person of ordinary skill in the art and is not to be limited to a special or customized meaning. “Electrically insulating material” may also refer to a dielectric material. The term specifically may refer, without limitation, to a material or combination of materials which prevent the transfer of electrical charges and which do not sustain a significant electrical current. Specifically, without limiting other possibilities, the at least one electrically insulating material may be or may comprise at least one insulating resin, such as insulating epoxy resins used in manufacturing electronic printed circuit boards; in particular it may comprise or be a thermoplastic material such as polycarbonate, polyester like polyethylene terephthalate (PET), polyvinyl chloride (PVC), polyurethane, polyether, polyamide, polyimide or a copolymer thereof, such as glycol modified polyethylene terephthalate, polyethylene naphthalate, polytetrafluoroethylene (PTFE) or alumina.

In the method and in the analyte sensor according to this disclosure, the substrate may comprise two opposing sides, a first side and a second side opposing the first side and at least one conductive material positioned on the first side of the substrate.

The term “conductive material,” as used herein, is a broad term and is to be given its ordinary and customary meaning to a person of ordinary skill in the art and is not to be limited to a special or customized meaning. The term specifically may refer, without limitation, to a conductive strip, layer, wire or other type of elongated electrical conductor. In certain embodiments, the conductive material forms at least one layer on the first side of the substrate.

More specifically, the term “conductive material” may refer, without limitation, to a material, which is conductive and hence capable of sustaining an electrical current, for example, the conductive material may comprise at least one material selected from the group consisting of: carbon; carbon paste; gold; copper; silver; nickel; platinum and palladium. Specifically, the conductive material may be or may comprise at least one metal, such as one or more of gold, copper, silver, nickel, palladium or platinum. Additionally or alternatively, the at least one conductive material may be or may comprise at least one conductive compound, such as at least one conductive organic or inorganic compound. Additionally or alternatively, the at least one conductive material may be or may comprise at least one nonmetallic conductive material, e.g., polyaniline, poly-3,4-ethylenedioxythiophene (PEDOT), carbon or carbon paste. Carbon paste specifically may relate to a material comprising carbon, a solvent such as diethylene glycol butyl ether, and at least a binder such as vinyl chloride co- and terpolymers. Preferably, the conductive material according to this disclosure may comprise gold and/or carbon; more preferably, the conductive material may consist of gold and/or carbon and/or carbon paste. Specifically the conductive material may comprise gold and a further material, for example, carbon.

Moreover, the conductive material may comprise at least one further layer of at least one further material; specifically the further layer may comprise a further conductive material. More specifically the further layer of the conductive material may comprise or may consist of carbon. The further material may be disposed on the first side. Using a further layer, in particular carbon, may contribute to efficient electron transfer by the conductive material.

The conductive material may have a thickness of at least about 0.1 μm, preferably of at least about 0.5 μm, more preferably of at least about 5 μm, specifically of at least about 7 μm, or at least about 10 μm. In the case where the conductive material comprises carbon or is carbon, the conductive material may specifically have a thickness of at least about 7 μm, more specifically of at least about 10 μm, for example, about 10 μm to 15 μm. Specifically, in the case where the conductive material is gold, the conductive material may have a thickness of at least about 100 nm, more specifically of at least about 500 nm.

A minimum thickness as specified above may be advantageous as it ensures proper electron transport. A thickness below the specified values is usually not sufficient for reliable electron transport. Even more specifically, the thickness should not exceed a value of about 30 μm in the case of carbon and a value of about 5 μm in the case of gold. If the thickness is too large, the overall thickness and hence the size of the analyte sensor may increase. Larger analyte sensor sizes are generally unwanted as they may cause difficulties when being implanted. Further, they may be less flexible, in particular in the case of carbon and/or they may be expensive, in particular in the case of gold.

The conductive material may be hydrophobic. For example, the contact angle of the conductive material with water may in the range from 60° to 140°, in particular about 100°, determined via microscopy, for example, using a Keyence VHX-100, with a water droplet volume of 5 μl.

The conductive material may further comprise a rough surface. A rough surface usually increases the efficiency of electron transfer. Further, it increases the hydrophobicity. A rough surface means that the surface may comprise unevenness. The depth of this unevenness may, for example, be in the range from 1 μm to 6 μm, such as about 3 μm, determined via light scanning microscopy, in particular via laser scanning microscopy. The distance between two rises in the rough surface may, for example, be in the range from 20 μm to 80 μm, such as about 40 μm, determined via light scanning microscopy, in particular via laser scanning microscopy.

The term “layer,” as used herein, is a broad term and is to be given its ordinary and customary meaning to a person of ordinary skill in the art and is not to be limited to a special or customized meaning. The term specifically may refer, without limitation, to an element of a layer setup of the analyte sensor. Specifically, the term “layer” may refer to an arbitrary covering of an arbitrary substrate, specifically of a flat substrate. The layer may specifically have a lateral extension exceeding its thickness by at least a factor of 2, at least a factor of 5, at least a factor of 10, or even at least a factor of 20 or more. Specifically, the analyte sensor may have a layer setup. The analyte sensor may comprise a plurality of layers such as the at least one conductive material, the at least one layer of the at least one sensing material, and optionally at least one membrane layer. One or more of the layers of the analyte sensor may comprise sub-layers. For example, a layer comprising the conductive material may comprise at least one further layer.

Step (b) of the method of this disclosure comprises applying a layer of an AgCl-containing composition onto the conductive material present on the first side of the substrate, wherein the layer of the AgCl-containing composition comprises an outer surface and an inner surface, wherein the outer surface faces away from the conductive material and wherein the inner surface is in contact with the conductive material.

The AgCl-containing composition may be may be applied by techniques known to those skilled in the art, using at least one coating process, specifically a wet-coating process, selected from the group consisting of: e.g., doctor-blading; dispensing; slot-dye coating; cannula-coating; and printing including screen printing, such as rotary screen printing.

The AgCl-containing composition may be an ink or a paste, particularly having a viscosity in the range from about 1000 mPas to about 10000 mPas when applied onto the conductive material on the first side of the substrate according to step (b). After application, a layer of the AgCl-containing composition positioned on the conductive material is obtained. The layer has an outer surface, which faces away from the conductive material, and an inner surface, which is in contact with the conductive material. Typically, the layer of the AgCl-containing composition has a thickness from about 1 μm to about 60 μm (dry thickness).

In certain embodiments, the AgCl-containing composition further comprises at least one binder. The binder may a non-conductive polymer, e.g., a polyester, a polyether, a copolymer of vinyl chloride (VC) and vinyl acetate (VAc), vinylester or vinylether, a polyvinylether, a polyvinylester, an acrylic resin, an acrylate or methacrylate, a styrene acrylic resin, a vinyl acetal, a thermoplastic olefin (TPO), a thermoplastic vulcanizate (TPV), a thermoplastic polyurethane (TPU), a thermoplastic copolyester (TPC), a polyamide, a thermoplastic elastomer (TPA), a styrenic block copolymer (TPS), an acrylonitrile butadiene styrene (ABS), a styrene-acrylonitrile resin (SAN), aa acrylonitrile styrene acrylate (ASA), a styrene butadiene copolymer (SB), a polystyrene (PS), a polyethylene (PE), an ethylene-vinyl acetate (EVA), a polypropylene (PP), a polybutylene (PB), a polyisobutene (PIB), a polyvinyl chloride (PVC), a polyvinyl alcohol (PVAL), a polylactic acid (PLA), particularly a polyvinyl chloride (PVC)-based polymer and/or a polyurethane-based polymer, e.g., a hydrophobic polyurethane-based polymer. The weight ratio of AgCl to the binder in the AgCl-containing composition may vary over a broad range and typically is from about 1:10 (w/w) to about 10:1 (w/w) or higher.

The AgCl of the AgCl-containing composition is typically comprised in solid form in the AgCl-containing composition. AgCl is preferably dispersed in the at least one binder.

In certain embodiments, the AgCl-containing composition further comprises elemental Ag when applied to the conductive material according to step (b), i.e., before step (c) of at least partially reducing the AgCl on the outer surface of the composition. For example, the weight ratio of Ag to AgCl in the AgCl-containing composition applied in step (b) may be from about 1/0.1 to about 1/5.

If elemental Ag is comprised in the AgCl-containing composition, the elemental Ag is typically comprised in solid form. Elemental Ag is preferably dispersed in the at least one binder together with AgCl.

During and after application of the AgCl-containing composition onto the conductive material in step (b), the distribution of AgCl and, optionally Ag, is homogeneously throughout the layer. Thus, preferably the inner and outer surface of the applied AgCl-containing composition have an identical composition.

According to step (c) of the method of this disclosure, the AgCl in the AgCl-containing composition is at least partially reduced on its outer surface wherein the outer surface faces away from the conductive material. Thereby elemental Ag is generated on the outer surface of the AgCl-containing composition. The reduction procedure is performed before implantation, i.e., outside the user's body.

The reduction of AgCl according to step (c) predominantly takes place at the outer surface of the AgCl-containing composition positioned on the conductive material on the first side of the substrate. Thus, the outer surface of the AgCl-containing composition has a content of AgCl, which is lower than the content of AgCl on the inner surface of the composition. Further, the outer surface of the AgCl-containing composition has a content of elemental Ag, which is higher than the content of Ag on the inner surface of the composition. In certain embodiments, the composition of the inner surface of the AgCl-containing composition, in particular the content of AgCl, and, if present, the content of elemental Ag remains essentially unchanged during step (c), e.g., a change of about 5% by weight or less or about 2% by weight or less based on the content before step (c).

In certain embodiments, an Ag layer is formed on the outer surface of the AgCl-containing composition. Throughout this layer, the AgCl has been substantially, i.e., at least about 90 mol-% or at least about 99 mol-% reduced to elemental Ag. The Ag layer on the outer surface of the AgCl-containing composition may have having a thickness from about 0.1 μm to about 5 μm.

In certain embodiments, an amount of about 0.2 μg/mm² to about 10 μg/mm² AgCl on the outer surface of the AgCl-containing composition is reduced.

Step (c) comprises an at least partial reduction of the AgCl on the outer surface of the AgCl-containing composition. According to this disclosure, not all of the AgCl in the whole layer of the AgCl-containing composition is reduced to Ag. In certain embodiments, about 1 mol-% to about 20 mol-% of the AgCl in the whole layer of the AgCl-containing composition is reduced to Ag.

The partial reduction of Ag in the AgCl-containing composition may be carried out at any point in time after application of the AgCl-containing composition to the substrate, i.e., at any time of the electrode manufacturing process, or the sensor manufacturing process, respectively. According to this disclosure, the partial reduction of Ag is carried out in vitro during the manufacturing process, i.e., outside a user's body.

In particular embodiments, the reduction is performed by an electrochemical treatment. The electrochemical treatment comprises applying a cathodic current to the AgCl-containing composition after application onto the conductive material in step (b). For example, the AgCl in the AgCl-containing composition may be reduced by an electrochemical treatment, wherein the substrate onto which the AgCl-containing composition has been applied is placed in a conductive aqueous solution, e.g., an electrolyte solution and polarized at a certain potential to invoke the reduction process. The conductive aqueous solution may comprise a salt such as NaCl, KCl and/or Na- or K-phosphate, any other salts, acids or bases. At least one external electrode, e.g., in form of a plate, a mesh or in any other form, is placed in the electrolyte solution together with the substrate comprising the AgCl-containing composition. Preferably, a three electrodes set-up is used with an additional external reference electrode for the electrochemical treatment. Preferably, a galvanostatic mode is used, while the electrochemical treatment is configured in order to draw a cathodic current from the AgCl-containing electrode, whereby the AgCl is reduced at a predefined rate depending from the strength of the cathodic current.

In alternative embodiments, the reduction of AgCl in the AgCl-containing composition is performed by a chemical treatment, e.g., by a treatment with a chemical reducing agent such as an aldehyde or uric acid under conditions where an at least partial reduction of AgCl on the outer surface of the AgCl-containing composition takes place.

Step (d) of the method of this disclosure comprises obtaining the partially reduced AgCl-containing electrode on the first side of the substrate. In particular embodiments, the electrode is a counter electrode and/or a reference electrode and/or a combined counter/reference electrode of an analyte sensor.

A further aspect of this disclosure is directed to a method for manufacturing an analyte sensor comprising manufacturing the electrode as described above and providing at least one working electrode. Typically, the working electrode is provided by applying a sensing material to a second conductive material positioned on a substrate.

The term “working electrode” as used herein, is a broad term and is to be given its ordinary and customary meaning to a person of ordinary skill in the art and is not to be limited to a special or customized meaning. The term specifically may refer, without limitation, to the electrode of the analyte sensor that is sensitive for the analyte. The working electrode may be disposed on a substrate. In particular, the working electrode comprises at least one conductive material, in the following “at least one second conductive material,” and at least one sensing material, wherein said at least one sensing material is applied onto the at least one second conductive material on the substrate. The second conductive material of the working electrode onto which the sensing material is applied may have features as described above for the conductive material onto which the electrode of this disclosure is applied.

In certain embodiments, the working electrode may be provided on the substrate, on which the partially reduced AgCl-containing electrode is positioned. Preferably, the working electrode is provided on the second side of the substrate, in particular on at least one second conductive material positioned on the second side of the substrate. Alternatively, the working electrode may be provided on the first side of the substrate together with the partially reduced AgCl-containing electrode, in particular on at least one second conductive material positioned on the first side of the substrate. In other embodiments, the working electrode may be provided on a different substrate, in particular on at least one second conductive material positioned on a different substrate.

Thus, the method of manufacturing an analyte sensor may comprise steps (a), (b), (c) and (d) as described above and further steps:
e) applying a sensing material to a substrate, in particular on at least one second conductive material positioned on the substrate, and
f) obtaining a working electrode of the analyte sensor on the substrate wherein the sensing material may comprise at least one enzyme, optionally at least one cross-linker and/or optionally at least one polymeric metal complex.

In particular embodiments, step (e) comprises applying a sensing material to the second side of the substrate, in particular on at least one second conductive material positioned on the second side of the substrate, and step (f) comprises obtaining a working electrode of the analyte sensor on the second side of the substrate. In these embodiments, the first side may oppose the second side.

In further particular embodiments, step (e) comprises applying a sensing material to the first side of the substrate, in particular on at least one second conductive material positioned on the first side of the substrate, and step (f) comprises obtaining a working electrode of the analyte sensor on the first side of the substrate. In these embodiments, the second conductive material is typically not in electrical contact with the conductive material onto which the electrode of this disclosure is positioned.

Reduction step (c) by means of an electrochemical treatment may be performed before or after manufacturing of the working electrode of the analyte sensor, preferably after the preparation of the working electrode. For example, an electrochemical reduction may be performed after cutting step (g).

Reduction step (c) by means of a chemical treatment is typically performed before manufacturing the working electrode of the analyte sensor.

The methods of manufacturing an electrode and manufacturing an analyte sensor may further comprise an additional step of drying at least one of the applied layers of the AgCl-containing composition and/or the sensing material. The drying step may take place at ambient temperature. Specifically, the sensing material may be dried at ambient temperature for about 10 minutes or less, or about 5 minutes or less, e.g., about 0.5 to about 10 minutes. The term “ambient temperature” as used herein is understood as a temperature specifically between 15° C. and 30° C., more specifically between 20° C. and 25° C.

According to step (e), a sensing material to a substrate, in particular on at least one second conductive material positioned on the substrate. The term “sensing material,” as used herein, is a broad term and is to be given its ordinary and customary meaning to a person of ordinary skill in the art and is not to be limited to a special or customized meaning.

The sensing material may comprise at least one enzyme; specifically the enzyme is capable of catalyzing a chemical reaction consuming at least the analyte; specifically the enzyme may be an H₂O₂ generating and/or consuming enzyme; even more specifically a glucose oxidase (EC 1.1.3.4), a hexose oxidase (EC 1.1.3.5), an (S)-2-hydroxy acid oxidase (EC 1.1.3.15), a cholesterol oxidase (EC 1.1.3.6), a glucose dehydrogenase (EC 1.1.1.47), a galactose oxidase (EC 1.1.3.9), an alcohol oxidase (EC 1.1.3.13), an L-glutamate oxidase (EC 1.4.3.11) or an L-aspartate oxidase (EC 1.4.3.16); even more specifically a glucose dehydrogenase (GOD) or a glucose oxidase (GOx) including any modifications thereof.

In certain embodiments, the sensing material comprises at least one crosslinker; the crosslinker may, for example, be capable of crosslinking at least part of the sensing material. Specifically the sensing material may comprise at least one crosslinker selected from UV-curable crosslinkers and chemical crosslinkers; more specifically the sensing material comprises a chemical crosslinker.

The term “chemical crosslinker” as used herein, is a broad term and is to be given its ordinary and customary meaning to a person of ordinary skill in the art and is not to be limited to a special or customized meaning. The term specifically may refer, without limitation, to a crosslinker that is capable of initiating a chemical reaction generating a crosslinked molecular network and/or a crosslinked polymer when exposed to heat. “Exposed to heat” may refer to being exposed to a temperature above 15° C., specifically to a temperature above 20° C.; more specifically to a temperature in the range from 20° C. to 50° C. and even more specifically to a temperature in the range from 20° C. to 25° C. More specifically, chemical crosslinkers may initiate crosslinking of the layer of the sensing material when exposed to heat.

Suitable chemical crosslinkers according to this disclosure are selected from: epoxide based crosslinkers, such as diglycidyl ethers like poly(ethylene glycol) diglycidyl ether (PEG-DGE) and poly(propylene glycol) diglycidyl ether; trifunctional short chain epoxides; anhydrides; diglycidyl ethers such as resorcinol diglycidyl ether, bisphenol, e.g., bisphenol A diglycidyl ether, diglycidyl 1,2-cyclohexanedicarboxylate, poly(ethylene glycol) diglycidyl ether, glycerol diglycidyl ether, 1,4-butanediol diglycidyl ether, poly(propylene glycol) diglycidyl ether, poly(dimethylsiloxane), diglycidyl ether, neopentyl glycol diglycidyl ether, 1,2,7,8-diepoxyoctane, 1,3-glycidoxypropyl-1,1,3,3-tetramethyldisioxane; triglycidyl ethers such as N,N-diglycidyl-4-glycidyloxyaniline, trimethylolpropane triglycidyl ether; tetraglycidyl ethers such as tetrakisepoxy cyclosiloxane, pentaerythritol tetraglycidyl ether, tetraglycidyl-4,4′-methylenebisbenzenamine.

The term “UV-curable” as used herein, is a broad term and is to be given its ordinary and customary meaning to a person of ordinary skill in the art and is not to be limited to a special or customized meaning. The term specifically may refer, without limitation, to the ability of a chemical substance, for example, a crosslinker, of initiating a photochemical reaction generating a crosslinked molecular network and/or a crosslinked polymer when irradiated by light in the UV spectral range. More specifically, UV-curable crosslinkers may initiate crosslinking of the layer of the sensing material when irradiated by UV light. Crosslinking may in particular be initiated as indicated herein below.

Suitable UV curable crosslinkers according to this disclosure include benzophenone, diazirine and azide. Particularly suitable UV-curable crosslinkers are, for example, selected from the group consisting of, benzophenone comprising crosslinkers, poly(di(2-hydroxy-3-aminobenzo-phenonepropylene) glycol), dibenzophenone 1,2-cyclohexane-dicarboxylate, bis[2-(4-azidosalicylamido)ethyl] disulfide, reaction products of the reaction of 4-aminobenzophenone with any one of the above for the chemical crosslinker described diglycidyl crosslinkers, triglycidyl crosslinkers and tetraglycidyl crosslinkers, an example of such a reaction product is 2,4,6,8-tetramethyl-2,4,6,8-tetrakis(2-hydroxy-3-aminpropylbenzophenone)-cyclotetrasiloxane, and reaction products of the reaction of 4-benzoylbenzoic acid N-succinimidyl ester with a diamine or a jeffamine.

Further, the sensing material may comprise at least one polymeric metal complex. The term “polymeric metal complex” specifically may refer, without limitation, to a material that may be or may comprise at least a polymeric material; specifically it may be or may comprise at least a polymeric material and at least a metal containing complex. The metal containing complex may be selected from the group of transition metal element complexes, specifically the metal containing complex may be selected from osmium-complexes, ruthenium-complexes, vanadium-complexes, cobalt-complexes, and iron-complexes, such as ferrocenes, such as 2-aminoethylferrocene. Even more specifically, the sensing material may comprise a polymeric transition metal complex as described, for example, in WO 01/36660 A2, the content of which is included by reference. In particular, the sensing material may comprise a modified poly (vinylpyridine) backbone loaded with poly(biimidizyl) Os complexes covalently coupled through a bidentate linkage. A suitable sensing material is further described in Feldmann et al, Diabetes Technology & Therapeutics, 5 (5), 2003, 769-779, the content of which is included by reference. Suitable sensing materials further may include ferrocene-containing polyacrylamide-based viologen-modified redox polymer, pyrrole-2,2′-azino-bis(3-ethylbenzthiazoline-6-sulfonic acid) (ABTS)-pyrene, naphthoquinone-LPEI. The polymeric transition metal complex may represent a redox mediator incorporated into a cross-linked redox polymer network. This is advantageous as it may facilitate electron transfer between the at least one enzyme or analyte and the conductive material. In order to avoid a sensor drift, the redox mediator and the enzyme may be covalently incorporated into a polymeric structure.

In certain embodiments, the sensing material comprises an enzyme capable of catalyzing a chemical reaction consuming at least the analyte, particularly an H₂O₂ generating and/or consuming enzyme, a crosslinker and a polymeric transition metal complex. Specifically, the sensing material may comprise at least a polymeric transition metal complex and GOx and a chemical crosslinker. More specifically, the sensing material may comprise a modified poly(vinylpyridine) backbone loaded with poly(bi-imidizyl) Os complexes covalently coupled through a bidentate linkage, GOx and a chemical crosslinker like poly(ethylene glycol) diglycidylether (PEG-DGE). Suitable further sensing materials are known to the person skilled in the art.

In an embodiment, the sensing material may comprise a polymeric material and MnO₂-particles, as well as the enzyme.

Suitable ways for initiating crosslinking depend on the type of crosslinker and are known by the person skilled in the art. Curing using UV-curable crosslinkers is generally induced by irradiation using UV light. As used herein, the term “UV light” generally refers to electromagnetic radiation in the ultraviolet spectral range. The term “ultraviolet spectral range” generally refers to electromagnetic radiation in the range of 1 nm to 380 nm, preferably light in the range of 100 nm to 380 nm. The curing usually may take place at room temperature.

The applying of the sensing material according to this disclosure is performed in at least one step, wherein a layer of a sensing material is applied using at least one coating process.

As further used herein, the term “coating process” may refer to an arbitrary process for applying at least one layer to at least one surface of an arbitrary object. The applied layer may cover the object, for example, the conductive material and/or the substrate completely or may only cover a part or parts of the object. The layer may be applied via a coating process wherein a material is provided, e.g., in a liquid form, exemplarily as a suspension or as a solution, and may be distributed on the surface. Specifically, the coating process may comprise a wet-coating process selected from the group consisting of: spin-coating; spray-coating; doctor-blading; printing; dispensing; slot-coating; dip-coating; and cannula-coating.

In step (f) of the method of this disclosure for the manufacturing of an analyte sensor, a working electrode of the analyte sensor is obtained on the substrate, preferably on the second side of the substrate, The term “to obtain at least one working electrode,” as used herein, is a broad term and is to be given its ordinary and customary meaning to a person of ordinary skill in the art and is not to be limited to a special or customized meaning. The term specifically may refer, without limitation, to forming and/or manufacturing the working electrode.

Step (f) may further comprise a partial removal of applied sensing material, e.g., by irradiating the sensing material with at least one laser beam, wherein at least the first portion of the applied sensing material is at least partially removed and wherein at least the second portion of the sensing material covering the at least one conductive material is preserved on the substrate to obtain at least one working electrode of the analyte sensor.

In certain embodiments, step (f) of the method of manufacturing an analyte sensor may further comprise an additional step of applying at least one membrane layer, the membrane layer at least partially covering the working electrode. The membrane layer generally may selectively allow for one or more molecules and/or compounds to pass, whereas other molecules and/or compounds are stopped by the membrane layer. Thus, the membrane layer is permeable for the at least one analyte to be detected. Thus, as an example, the membrane layer may be permeable for one or more of glucose, lactate, cholesterol or other types of analytes. The at least one membrane layer may hence function as a diffusion barrier that controls diffusion of the analyte from the exterior, e.g., the body fluid surrounding the analyte sensor, to the sensing material, i.e., the enzyme molecules in the sensing material. In addition, the at least one membrane layer may function as a biocompatibility membrane layer as mentioned elsewhere herein.

The membrane layer, as an example, may have a thickness sufficient for providing mechanical stability. The at least one membrane layer specifically may have a thickness of about 1 μm to about 150 μm. For the at least one membrane layer, as outlined herein, several materials may be used, standalone or in combination. Thus, as an example, the membrane layer specifically may comprise one or more of a polymeric material, specifically a polyvinyl pyridine based copolymer, a polyurethane; a hydrogel; a polyacrylate; a methacrylate-acrylate copolymer or block-copolymer; among which polyvinyl pyridine based copolymers are particularly suitable. These types of membranes are generally known in the art. Moreover, the membrane layer may comprise a crosslinker, specifically a chemical crosslinker or a UV-curable crosslinker, e.g., as described above.

In step (f), in addition to the at least one membrane layer, at least a second membrane layer may be applied. Said second membrane layer may be a biocompatibility membrane layer.

The biocompatibility layer may have a thickness of from about 1 μm to about 10 μm, in an embodiment of from about 3 μm to about 6 μm. More specifically, the biocompatibility layer covers the analyte sensor at least partly or completely. Even more specifically, the biocompatibility layer may be the outmost layer of the analyte sensor. The biocompatibility membrane layer may be or may comprise at least one of the following materials: polyvinyl pyridine based copolymers, methacrylate based polymers and copolymers, acrylamide-methacrylate based copolymers, biodegradable polysaccharides such as hyaluronic acid (HA), agarose, dextran and chitosan.

The at least one membrane layer and/or the biocompatibility membrane layer may be applied by techniques known to those skilled in the art, using at least one coating process, specifically a wet-coating process, selected from the group consisting of: e.g., spin-coating; spray-coating; doctor-blading; printing; dispensing; slot-coating; dip-coating. A preferred wet-coating process is dip-coating or spray-coating.

The method according to this disclosure may further comprise at least one diffusion step wherein, in the diffusion step the crosslinker comprised in the membrane layer may at least partially diffuse into the sensing material. Diffusion may occur during applying the membrane layer to the sensing material. The diffusion of the crosslinker into the sensing material may allow for at least partial crosslinking of the sensing material independent of the amount of crosslinker in the sensing material during step (e) of applying the sensing material to the substrate.

In the method according to this disclosure, the diffusion step may further comprise a swelling of at least a part of the sensing material. The term “swelling” as used herein is a broad term and is to be given its ordinary and customary meaning to a person of ordinary skill in the art and is not to be limited to a special or customized meaning. The term specifically may refer, without limitation, to the binding of water and/or to the binding of water-soluble solvent such as ethanol, methanol, acetone to a material, specifically to the binding of water and/or of water-soluble solvent to the sensing material. Due to the uptake of water and/or the uptake of water-soluble solvent into the sensing material, diffusion of the crosslinker into the sensing material may advantageously be enabled which may be required for efficient crosslinking. Swelling may moreover refer to the uptake of water from the membrane layer.

To allow for sufficient swelling in the method according to this disclosure, the polymeric material in the sensing material may be capable of taking up of at least 10 wt.-% of water and/or solvent from the membrane layer based on the dry weight of the polymeric material within a time frame of several minutes, e.g., 1 to 15 minutes, more specifically at least 20 wt.-%, even more specifically at least 30 wt.-%, even more specifically up to 90 wt.-%.

This swelling and/or uptake of water and/or solvent is advantageous as diffusing of the crosslinker from the membrane layer into the sensing material may thereby be enabled.

The method for obtaining an analyte sensor of this disclosure may comprise at least one of the further steps:
g) cutting at least one substrate into predetermined portions and
h) confectioning the analyte sensor.

In step (g), at least one substrate is cut into predetermined portions. Typically, the predetermined portions have a size suitable as an analyte sensor, particularly as an implantable analyte sensor, e.g., a length of less than about 50 mm, such as a length of about 30 mm or less, e.g., a length of 5 mm to 30 mm and/or a width of about 200 μm to 1000 μm, more precise from 500 μm to 700 μm.

In certain embodiments, a portion of the substrate includes both an electrode of this disclosure and a working electrode as described above. Cutting may be performed by laser cutting and/or die cutting.

In step (h), the analyte sensor is confectioned. Typically, confectioning comprises making the analyte sensor ready for use and may involve sterilizing, and/or packaging, and/or connecting to an electronics unit.

Particularly, the analyte sensor according to this disclosure may be fully or a partially implantable and may, thus, be adapted for performing the detection of the analyte in the body fluid in a subcutaneous tissue, in particular, in an interstitial fluid. Other parts or components may remain outside of the body tissue. For example, as used herein, the terms “implantable” or “subcutaneous” refer to be fully or at least partly arranged within the body tissue of the user. For this purpose, the analyte sensor may comprise an insertable portion, wherein the term “insertable portion” may generally refer to a part or component of an element configured to be insertable into an arbitrary body tissue. The insertable portion comprises the working electrode and at least one further electrode, which is a partially reduced AgCl-containing electrode of this disclosure, e.g., as a counter, reference and/or counter/reference electrode. In certain embodiments, the working electrode is positioned on the second side of the substrate, the partially reduced AgCl-containing electrode is positioned on the first side of the substrate and all electrodes are positioned on the insertable portion. The part of the sensor, which is not inserted, is the upper part of the sensor, which comprises the contacts to connect the sensor to an electronics unit.

The AgCl-containing electrode may be comprised in an analyte sensor, typically as a counter electrode and/or a reference electrode and/or a combined counter/reference electrode. The analyte sensor further comprises additionally a working electrode comprising a layer of sensing material, which is typically absent from the AgCl containing electrode and/or any further electrodes, e.g., the counter electrode and/or the reference electrode and/or the combined counter/reference electrode.

The working electrode is sensitive for the analyte to be measured at a polarization voltage, which may be applied between a working electrode and at least one further electrode, e.g., one counter/reference electrode, in particular the electrode of this disclosure, wherein the polarization voltage may be regulated by a potentiostat. The potentiostat may be part of the electronics unit. A measurement signal may be provided as an electric current between the counter electrode and the working electrode. A separate counter electrode may be absent and a pseudo reference electrode may be present, which may also work as a counter electrode. Thus, an analyte sensor typically may comprise a set of at least two, in an embodiment a set of three electrodes. Particularly, the sensing material is present in the working electrode only.

Preferably, the insertable portion may fully or partially comprise a biocompatible surface, which may have as little detrimental effects on the user or the body tissue as possible, at least during typical durations of use. For this purpose, the insertable portion may be fully or partially covered with at least one biocompatibility membrane layer, such as at least one polymer membrane, for example, a gel membrane which, on one hand, may be permeable for the body fluid or at least for the analyte as comprised therein, and may on the other hand be impermeable for compounds comprised in the analyte sensor, in particular in the working electrode, thus preventing a migration thereof into the body tissue. Further details regarding the biocompatibility membrane layer are disclosed elsewhere herein.

Further, the term “analyte” as used herein is a broad term and is to be given its ordinary and customary meaning to a person of ordinary skill in the art and is not to be limited to a special or customized meaning. The term specifically may refer, without limitation, to an arbitrary element, component or compound which may be present in a body fluid and the concentration of which may be of interest for a user. Specifically, the analyte may be or may comprise an arbitrary chemical substance or chemical compound, which may take part in the metabolism of the user, such as at least one metabolite. As an example, the at least one metabolite may be selected from the group consisting of glucose, cholesterol, triglycerides, lactate; more specifically the analyte may be glucose. Additionally or alternatively, however, other types of analytes and/or any combination of analytes may be determined.

Specifically, the analyte sensor comprises a partially reduced AgCl-containing electrode positioned on the at least one first side of the substrate. The partially reduced AgCl-containing electrode comprises a layer of an AgCl-containing composition comprising an outer surface, wherein the outer surface faces away from the conductive material. According to this disclosure, the AgCl on the outer surface of the layer of the AgCl-containing composition is at least partially reduced and elemental Ag is present on the outer surface of the AgCl-containing composition. In certain embodiments, the partially reduced AgCl-containing electrode may be at least one of a reference electrode and a counter electrode. In an embodiment, the partially reduced AgCl-containing electrode is a combined counter/reference electrode.

Further, this disclosure relates to an analyte sensor comprising at least one partially reduced AgCl-containing electrode as described above.

The analyte sensor as described herein may in particular be obtainable by the method according to this disclosure for the preparation of an partially reduced AgCl-containing electrode on a substrate, e.g., as a counter electrode or a reference electrode or a combined counter/reference electrode, and a step of providing at least one working electrode.

Moreover, this disclosure relates to the use of the analyte sensor for detecting at least one analyte in a sample, specifically in a sample of a body fluid. More particularly, the analyte sensor is a sensor for continuous glucose measurement.

As used herein, the term “body fluid” relates to all bodily fluids of a subject known to comprise or suspected to comprise the analyte of this disclosure, including interstitial fluid, blood, plasma, lacrimal fluid, urine, lymph, cerebrospinal fluid, bile, stool, sweat, and saliva. Generally, an arbitrary type of body fluid may be used. Preferably, the body fluid is a bodily fluid which is present in a body tissue of a user, such as in the interstitial tissue. Thus, as an example, the body fluid may be selected from the group consisting of blood and interstitial fluid. However, additionally or alternatively, one or more other types of body fluids may be used. The body fluid generally may be contained in a body tissue. Thus, generally, the detection of the at least one analyte in the body fluid may preferably be determined in vivo.

The term “sample” is understood by the skilled person and relates to any sub-portion of a bodily fluid. Samples can be obtained by well-known techniques including, e.g., venous or arterial puncture, epidermal puncture, and the like.

Moreover, this disclosure relates to a method for measuring an analyte in a sample comprising the analyte sensor described herein above.

The methods for measuring of an analyte of this disclosure, in particular, may be in vivo methods. Alternatively, the method of this disclosure may also encompass measuring of an analyte under in vitro conditions, e.g., in a sample of a body fluid obtained from a subject, particularly from a human subject. Specifically, said method may not comprise diagnosis of disease based on said measurement.

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of this disclosure is not restricted by the preferred embodiments.

Summarizing and without excluding further possible embodiments, the following embodiments may be envisaged:

Embodiment 1: A method for manufacturing an electrode of an analyte sensor, the method comprising the steps:
a) providing a substrate comprising
   a first side and a second side, and
   at least one conductive material positioned on the first side of the substrate,
b) applying a layer of an AgCl-containing composition onto the conductive material, wherein the layer of the AgCl-containing composition comprises an outer surface and an inner surface, wherein the outer surface faces away from the conductive material and wherein the inner surface is in contact with the conductive material, and
c) at least partially reducing AgCl on the outer surface of the layer of the AgCl-containing composition, thereby forming elemental Ag on the outer surface, and
d) obtaining the electrode of the analyte sensor on the first side of the substrate.

Embodiment 2: The method of Embodiment 1, wherein the AgCl-containing composition applied in step b) further comprises at least one binder and/or elemental Ag.

Embodiment 3: The method of Embodiment 2, wherein the binder is a non-conductive polymer, particularly a PVC-based polymer and/or a polyurethane-based polymer.

Embodiment 4: The method of Embodiment 2 or 3, wherein the binder is a hydrophobic polyurethane-based polymer.

Embodiment 5: The method of any one of the Embodiments 2-4, wherein the weight ratio of AgCl to the binder in the AgCl-containing composition applied in step b) is from about 1:10 (w/w) to about 10:1 (w/w) or higher.

Embodiment 6: The method of any one of Embodiments 2-5, wherein the weight ratio of Ag to AgCl in the AgCl-containing composition applied in step b) is from about 1:0.1 to about 1:5.

Embodiment 7: The method of any one of Embodiments 1-6, wherein the AgCl-containing composition applied in step b) is an ink or a paste, particularly having a viscosity 1000 mPas and 10000 mPas.

Embodiment 8: The method of any one of Embodiments 1-7, wherein the at least one conductive material is selected from gold, carbon, carbon paste and any combination thereof.

Embodiment 9: The method of Embodiment 8, wherein the at least one conductive material comprises at least two different layers, particularly a gold layer and a carbon layer.

Embodiment 10: The method of any one of Embodiments 1-9, wherein in step c) the AgCl is reduced by a chemical treatment and/or by an electrochemical treatment.

Embodiment 11: The method of Embodiment 10, wherein in step c) the AgCl is reduced by an electrochemical treatment in a conductive aqueous solution using an external electrode.

Embodiment 12: The method of any one of Embodiments 1-11, wherein in step c) about 1 mol-% to about 20 mol-% of the AgCl in the whole layer of the AgCl-containing composition are reduced to Ag.

Embodiment 13: The method of any one of Embodiments 1-12, wherein in step c) an amount of about 0.2 μg/mm² to about 10 μg/mm² AgCl on the outer surface of the AgCl-containing composition is reduced.

Embodiment 14: The method of any one of Embodiments 1-13, wherein in step c) a layer of Ag having a thickness from about 0.1 μm to about 5 μm is formed on the outer surface of the AgCl-containing composition, particularly wherein at least about 90 mol-% or at least about 99 mol-% of the AgCl in said layer on the outer surface are reduced to elemental Ag.

Embodiment 15: The method of any one of Embodiments 1-14, wherein the electrode is used as a reference electrode, a counter electrode and/or a combined counter/reference electrode on an analyte sensor.

Embodiment 16: The method of any one of Embodiments 1-15, wherein the substrate comprises at least one conductive material positioned on the second side of the substrate.

Embodiment 17: A method for manufacturing an analyte sensor, comprising manufacturing an electrode according to any one of Embodiments 1-15 and providing a working electrode.

Embodiment 18: The method of Embodiment 17 further comprising the steps:
e) applying a sensing material to a substrate, in particular on at least one second conductive material positioned on the substrate, and
f) obtaining a working electrode of the analyte sensor on the substrate wherein the sensing material may comprise at least one enzyme, optionally at least one cross-linker and/or optionally at least one polymeric metal complex.

Embodiment 19: The method of Embodiment 18, wherein step (e) comprises applying a sensing material to the second side of the substrate, in particular on at least one second conductive material positioned on the second side of the substrate, and step (f) comprises obtaining a working electrode of the analyte sensor on the second side of the substrate.

Embodiment 20: The method of Embodiment 19, wherein the first side opposes the second side.

Embodiment 21: The method of Embodiment 18, wherein step (e) comprises applying a sensing material to the first side of the substrate, in particular on at least one second conductive material positioned on the first side of the substrate, and step (f) comprises obtaining a working electrode of the analyte sensor on the first side of the substrate.

Embodiment 22: The method of any one of Embodiments 18-21, wherein the sensing material comprises at least one enzyme, optionally at least one crosslinker and/or optionally at least one polymeric metal-containing complex.

Embodiment 23: The method of any one of Embodiments 18-22, wherein the enzyme is a glucose dehydrogenase (GOD) or a glucose oxidase (GOx).

Embodiment 24. The method of any one of Embodiments 18-23, further comprising at least one of the steps:
g) cutting the substrate into predetermined portions and
h) confectioning the analyte sensor.

Embodiment 25: An electrode of an analyte sensor obtainable by the method of any one of Embodiments 1-16.

Embodiment 26: An analyte sensor obtainable by the method of any one of Embodiments 17-24.

Embodiment 27: An analyte sensor comprising:
(i) a substrate comprising
   a first side and a second side, and
   at least one conductive material positioned on the first side of the substrate,
(ii) an electrode positioned on the at least one conductive material, wherein the electrode comprises an outer surface and an inner surface, wherein the outer surface faces away from the conductive material and wherein the inner surface is in contact with the conductive material, and wherein the AgCl on the outer surface of the layer of the AgCl-containing composition is at least partially reduced and elemental Ag is present on the outer surface of the AgCl-containing composition, and
(iii) at least one working electrode.

Embodiment 28: The analyte sensor of Embodiment 26 or 27, wherein the content of AgCl on the outer surface of the AgCl-containing composition is less than the content of AgCl on the inner surface of the AgCl-containing composition.

Embodiment 29: The analyte sensor of any one of Embodiments 26-28, wherein a layer of Ag having a thickness from about 0.1 μm to about 5 μm is present on the outer surface of the AgCl-containing composition, particularly wherein at least about 90 mol-% or at least about 99 mol-% of the AgCl in this layer have been reduced to elemental Ag.

Embodiment 30: The analyte sensor of any one of Embodiments 26-29, wherein the substrate (i) further comprises at least one second conductive material positioned on the second side of the substrate.

Embodiment 31: The analyte sensor of any one of Embodiments 26-30, wherein the working electrode is positioned on the second side of the substrate.

Embodiment 32: The analyte sensor of any one of Embodiments 26-29, wherein the substrate (i) further comprises at least one second conductive material positioned on the first side of the substrate.

Embodiment 33: The analyte sensor of any one of Embodiments 26-29 or 32, wherein the working electrode is positioned on the first side of the substrate.

Embodiment 34: The analyte sensor of any one of Embodiments 26-33, wherein the first side opposes the second side.

Embodiment 35: The analyte sensor of any one of Embodiments 26-36, wherein the working electrode comprises at least one sensing material, and wherein the sensing material comprises at least one enzyme, optionally at least one crosslinker and/or optionally at least one polymeric metal-containing complex.

Embodiment 36: The analyte sensor of Embodiment 35, wherein the enzyme is a glucose dehydrogenase (GOD) or a glucose oxidase (GOx).

Embodiment 37: The analyte sensor of any one of Embodiments 26-36, which is a two electrode-sensor comprising one partially reduced AgCl-containing electrode and one working electrode.

Embodiment 38: The analyte sensor of any one of Embodiments 26-37, which is an amperometric sensor.

Embodiment 39: The analyte sensor of any one of Embodiments 26-38, which is sterilized and/or packaged.

Embodiment 40: Use of an analyte sensor of any one of Embodiments 26-39 for detecting at least one analyte.

Embodiment 41: A method for determining an analyte in a sample using the analyte sensor of any one of Embodiments 26-39.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1 shows a current curve recorded in vivo from a GOD-based amperometric analyte sensor of the prior art.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

### Example

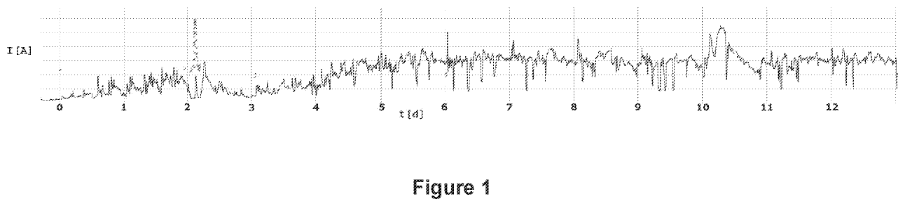

FIG. 1 shows a typical current curve I in ampere (A) over a time t of twelve days (d) recorded in vivo from a amperometric analyte sensor comprising a working electrode comprising a GOD-containing sensing material and a counter/reference electrode of the prior art, which is manufactured by applying an AgCl-containing composition onto a conductive material on a substrate. The sensor shows diminished current shortly after operation start up to few days. This diminished current shows the so-called run-in time of the sensor. Only after the run-in time, when the sensor shows sufficiently high current, a reliable measurement is possible.

According to this disclosure, an electrochemical treatment of the counter/reference electrode is provided in order to convert the AgCl on the outer surface of the AgCl-containing composition to elemental silver before the sensor is implanted into a user's body.

Thereby, from the very beginning of the insertion of the analyte sensor reliable measurements can be obtained.

The electrochemical treatment may include connection of the Ag/AgCl-containing electrode to a galvanostat, wherein a further external electrode in form of a plate, mesh or any other form is used. An additional reference electrode may be used. All electrodes may be placed in an electrolyte solution, such as 100 mM KCl or a buffered solution, like phosphate-buffered saline (PBS). The galvanostat is configured to draw a cathodic current from the Ag/AgCl-containing electrode, which means, the AgCl is being reduced at a predefined rate. The reduction rate corresponds to the value of the cathodic current drawn from the Ag/AgCl-containing electrode and depends on the concrete sensor construction.

In an exemplary and non-limiting embodiment, a charge of about 0.00216 C may be drawn from the Ag/AgCl-containing electrode in order to reduce the AgCl at the outer surface of the AgCl-containing composition. For instance, 0.00216 C can be drawn with 1 h, if the pre-set current is 600 nA.

The electrochemical treatment may be performed before or after application of the sensing material to the working electrode. The method of this disclosure is not limited to flat analyte sensors, but is applicable to any AgCl-containing electrode.

An Ag/AgCI containing electrode was manufactured by applying an AgCl-containing composition on a sensor substrate as a layer with a thickness of 15 μm, a width of 400 μm and a length of 4 mm. The layer was dried and covered by photoresist with four areas as squares (175 μm×175 μm) remaining uncoated. The dried AgCl-containing composition had the following composition: 19% by weight Ag, 65% by weight AgCl, 16% by weight of a polyvinylchloride-based binder (available under the trademark VINNOL by Wacker Chemie AG), in each case based on the total weight of the dried AgCl-containing composition. The total amount on the surface was therefore 18 μg Ag, 61.3 μg AgCl and 15 μg binder. About 4 to 5 μg AgCl were reduced, which corresponds to about 10% of the overall content of AgCl.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

1. A method for manufacturing an electrode of an analyte sensor, the method comprising:
a) providing a substrate having a first side and a second side and a conductive material positioned on the first side;
b) applying a layer of an AgCl-containing composition onto the conductive material, wherein the layer of the AgCl-containing composition comprises an outer surface that faces away from the conductive material and an inner surface that contacts the conductive material;
c) at least partially reducing AgCl on the outer surface and thereby forming elemental Ag on the outer surface; and
d) obtaining the electrode of the analyte sensor on the first side of the substrate.

2. The method of claim 1, wherein the AgCl-containing composition applied in step b) further comprises at least one binder and/or elemental Ag.

3. The method of claim 2, wherein the binder comprises a non-conductive polymer.

4. The method of claim 3, wherein the binder is one or more of a PVC-based polymer and a polyurethane-based polymer.

5. The method of claim 4, wherein the binder is a hydrophobic polyurethane-based polymer.

6. The method of claim 1, wherein the conductive material is selected from the group consisting of gold, carbon, carbon paste and combinations thereof.

7. The method of claim 6, wherein the conductive material comprises at least two different layers.

8. The method of claim 7, wherein the conductive material comprises a gold layer and a carbon layer.

9. The method of claim 1, wherein in step c) the AgCl is reduced by a chemical treatment and/or by an electrochemical treatment.

10. The method of claim 1, wherein in step c) an amount of about 0.2 μg/mm² to about 10 μg/mm² AgCl on the outer surface is reduced.

11. The method of claim 1, wherein in step c) a layer of Ag having a thickness from about 0.1 μm to about 5 μm is formed on the outer surface of the AgCl-containing composition.

12. The method of claim 11, wherein about 90 mol-% to about 99 mol-% of the AgCl in the layer of Ag is reduced to elemental Ag.

13. The method of claim 1, wherein the electrode is used as a reference electrode, a counter electrode and/or a combined counter/reference electrode of an analyte sensor.

14. A method for manufacturing an analyte sensor, comprising manufacturing an electrode according to claim 1 and providing a working electrode.

15. The method of claim 14, wherein providing the working electrode comprises applying a sensing material to the substrate and obtaining a working electrode of the analyte sensor on the substrate.

16. The method of claim 15, wherein the sensing material comprises at least one enzyme.

17. The method of claim 16, wherein the sensing material comprises a cross-linker and/or a polymeric metal complex.

18. The method of claim 17, wherein the enzyme is a glucose dehydrogenase (GOD) or a glucose oxidase (GOx).

19. An analyte sensor, comprising:
a substrate having a first side and a second side and a conductive material positioned on the first side;
an electrode positioned on the conductive material, wherein the electrode comprises a layer of an AgCl-containing composition and has an outer surface facing away from the conductive material and an inner surface in contact with the conductive material, and wherein the AgCl on the outer surface is at least partially reduced and elemental Ag is present on the outer surface; and
a working electrode.

20. The analyte sensor of claim 19, wherein the content of AgCl on the outer surface is less than the content of AgCl on the inner surface.

21. The analyte sensor of claim 19, wherein the working electrode at least partially covers the second side of the substrate, wherein the working electrode comprises at least one sensing material.

22. The analyte sensor of claim 21, wherein the sensing material comprises an enzyme.

23. The analyte sensor of claim 22, wherein the enzyme is a glucose dehydrogenase (GOD) or a glucose oxidase (GOx).
